(19) United States
(12) Patent Application Publication
(10) Pub. No.: US 2026/0126442 A1
(43) Pub. Date: May 7, 2026

(54) METHODS FOR MONITORING IMMUNE STATUS OF A POPULATION

(71) Applicant: THE UNIVERSITY OF NORTH CAROLINA AT CHARLOTTE, Charlotte, NC (US)

(72) Inventors: Cynthia GIBAS, Charlotte, NC (US); Jessica SCHLUETER, Concord, NC (US); William TAYLOR, Gastonia, NC (US); Marie LittleFawn AGAN, Mooers, NY (US)

(21) Appl. No.: 19/118,196

(22) PCT Filed: Oct. 24, 2023
(86) PCT No.: PCT/US2023/077616

§ 371 Date: Apr. 3, 2025

Related U.S. Application Data
(60) Provisional application No. 63/419,205, filed on Oct. 25, 2022.

(51) Int. Cl. G01N 33/569

(52) U.S. Cl. CPC G01N 33/56983; G01N 2333/165; G01N 2469/20

(57) ABSTRACT

The present invention relates to methods for monitoring the immune status of a population by detecting and/or quantifying antibodies present in wastewater. The invention further provides methods and kits for monitoring the presence of an antibody to SARS-CoV-2 or a variant thereof within a population by detecting and/or quantifying SARS-CoV-2-specific antibodies in wastewater.

### STATEMENT OF PRIORITY

The present invention claims the benefit, under 35 U.S.C. § 119 (e), of U.S. Provisional Application No. 63/419,205, filed Oct. 25, 2022, the entire contents of which are incorporated by reference herein.

### STATEMENT REGARDING ELECTRONIC FILING OF A SEQUENCE LISTING

A Sequence Listing in XML format, entitled 9812-11WO_ST26.xml, 29,476 bytes in size, generated on Oct. 21, 2023 and filed herewith, is hereby incorporated by reference in its entirety for its disclosures.

### FIELD OF THE INVENTION

The present invention relates to the field of wastewater surveillance for monitoring immune status and infectious disease in populations.

### BACKGROUND OF THE INVENTION

Seroprevalence surveillance plays a critical role in detection and epidemiology of infectious disease outbreaks (Choisy, et al. (2019) Open Forum Infect. Dis. 6: ofz030; Wilson et al. (2012) CMAJ 184: E70-6). Serological evidence of infection can also be used as an indicator of likelihood of zoonotic spillover and precursor to emergence of novel diseases (Li et al. (2019) Biosaf. Health 1:84-90). The onset of the COVID-19 pandemic led to a call for a Global Immunological Observatory (Mina et al. (2020) Elife 9: e58989) that would provide information about susceptibility of populations based on aggregate serological data, allowing for public health action in advance of waves of infection. However, there are several requirements which are barriers to large-scale data collection and the achievement of such an aim. One of these is the need to collect large numbers of serological samples from individual patients, which implies the need for patient participation, or to reliably obtain material from clinical discards. Such requirements are especially challenging in low and middle income countries (Haselbeck et al. (2022) Pathogens 11 (7) 732) but can pose a challenge even in resource-rich areas. In the case of the SARS-CoV-2 virus, a massive amount of genomic surveillance data was collected as governments required testing and made free testing available, but as pandemic mitigation measures have declined or ended, clinical test discards for sequencing have become difficult to obtain and surveillance by genomic sequencing has declined significantly. In turn, health departments have turned to sequencing and viral variant identification out of wastewater as a non-invasive method that can be conducted independent of patient choices about treatment and testing.

Wastewater surveillance has proven useful at both the regional scale for detection of SARS-CoV-2 infection trends (Peccia et al. (2020) Nat. Biotechnol. 38:1164-1167; Wu et al. (2022) Sci. Total Environ. 805:150121; Weidhaas et al. (2021) Sci. Total Environ. 775:145790), and at the local scale for guiding rapid response to emerging outbreaks (Gibas et al. (2021) Sci. Total Environ. 782:146749; Johnson et al. (2022) FEMS Microbes 24:3:xtac024). The main modes of wastewater surveillance during the COVID-19 pandemic have been quantitative detection and variant sequencing of viral RNA isolated from wastewater. However, the potential for monitoring other immunologically relevant molecules in wastewater has not been as extensively explored.

Immunoassay systems have been widely used as a biotechnology tool for detection of SARS-CoV-2 RNA (Fourati et al. (2022) J. Clin. Virol. 146:105048; Drain et al. (2021) Infect. Dis. Ther. 10:753-61) and virus in clinical samples. Samples for immunoassays are typically collected from serum (Guerrero-Esteban et al. (2022) Talanta 247:123543), but there have also been recent advances in use of immunosensors targeting the viral antigen protein in wastewater (Thongpradit et al. (2022) Int. J. Environ. Res. Public Health 19 (13): 7783; Lu et al. (2021) Sci. Total Environ. 77:146239). Antibody measurements from fecal samples have been described as a non-invasive means to rapidly monitor the immune status of wildlife (Watt et al. (2016) Ecol. Evol. 6:56-67). Similarly, measurements from fecal samples are being used for studying various aspects of human health (Frehn et al. (2014) PLoS ONE 9: e106750; Lin et al. (2018) J. Transl. Med. 16:359), as fecal antibody levels are correlated with antibody levels in serum (Kolmannskog & Haneberg (1985) Int. Arch. Allergy Appl. Immunol. 76:133-137), and have been demonstrated as a potential biomarker for detection of viral (Niedrig et al. (2018) BMC Infect. Dis. 18:707) and parasitic infections (Nagaoka et al. (2021) Vaccines (Basel) 9 (7): 778) in individual human urine samples.

Infection by SARS-CoV-2 elicits the secretion of mucosal IgG and IgA antibodies (Lehmann et al. (2021) Mucosal Immunol. 14:1381-1392). The IgA response waxes and wanes rapidly, being detectable in serum within days of infection, before falling below the limit of detection within weeks. In contrast, the signal of the IgG response in serum rises more slowly and may persist for months (Townsend et al. (2021) Lancet Microbe 2: e666-e675), potentially remaining above the limit of detection even longer (FIG. 1). Coronavirus-specific antibodies are also known to be recoverable from fecal samples in animal models (Decaro et al. (2004) Clin. Diagn. Lab. Immunol. 11:102-105). In general, the focus of research in this area has been on detection of antibodies from individual clinical specimens.

Neutralization activity against SARS-CoV-2 is directly correlated with measurements of anti-SARS-CoV-2 IgG or IgA antibodies (Dolscheid-Pommerich et al. (2022) J. Med. Virol. 94:388-392). Short-term longitudinal studies of SARS-CoV-2 neutralizing antibodies in naturally infected, vaccinated, or boosted individuals have made clear that antibody protection against future infection wanes within a period of months (Shrotri et al. (2022) Lancet Healthy Longev. 3: e470-e480; Townsend et al. (2022) Proc. Natl. Acad. Sci. USA 119: e2204336119). As governmental interventions ebb globally and rates of booster vaccine uptake continue to decline, there is little doubt that COVID-19 will transition to an endemic disease (Phillips (2021) Nature 590:382-384). Understanding the susceptibility of populations to future waves of infections is critical for mitigating an endemic state that results in unacceptable morbidity and mortality. The present invention addresses this need by expanding the scope of surveillance to include detection of antibody levels to pathogens in wastewater thereby providing a proactive approach that can identify populations at risk of future infection. Decrease of community-wide antibody levels in a sewershed would reveal waning of a durable immune response in the population after episodes of widespread infection or after seasonal vaccination campaigns.

### SUMMARY OF THE INVENTION

The present invention provides for the estimation of the immune status of a population by surveillance of wastewater. The invention is based upon the unexpected finding that human IgG and IgA antibodies are detectable in community wastewater, are not degraded by proteases, and are present in sufficient quantities to be measured. In addition, the human IgG and IgA antibodies in wastewater samples, while being at substantially lower concentrations than found in typical serum samples, are detectable in concentrated wastewater samples, e.g., via an ELISA assay, and are therefore indicative of an immune response to an antigen, e.g., an antigen of a pathogenic virus, bacterium, fungus or parasite. In particular, the results herein show that specific SARS-CoV-2 antibodies can be detected in wastewater thereby allowing for monitoring of antibody responses to SARS-CoV-2 antigens and retroactive studies of archived samples collected during the SARS-CoV-2 pandemic. Unlike measurements of viral abundance, which reflect viral shedding by already-infected individuals, antibody levels detected across the community may be an indicator for a proactive public health approach. Thus, adding the ability to assay antibody levels in wastewater in tandem with viral detection and sequencing increases the power of wastewater surveillance to provide data critical for public health policy and decision making, e.g., identifying populations with waning immunity and boosting with updated vaccines that reflect circulating variants.

Accordingly, in one aspect, the invention provides a method of detecting the presence of an antibody to an antigen of interest within a population comprising (a) contacting a wastewater sample from a population with an antigen of interest, and (b) detecting binding of the antibody to the antigen of interest, thereby detecting the antibody to the antigen of interest within the population.

In another aspect, the invention provides a method of determining immune status of a population to a pathogen comprising (a) contacting a wastewater sample from a population with at least one antigen from the pathogen; and (b) detecting binding of antibodies present in the wastewater sample to the at least one antigen from the pathogen, thereby determining the immune status of the population to the pathogen.

In a further aspect, the invention provides a method of monitoring the presence of an antibody to an antigen of SARS-CoV-2 or variant thereof within a population, comprising (a) concentrating a wastewater sample from a population; (b) detecting the presence of an antibody that binds to an antigen of SARS-CoV-2 or variant thereof in the concentrated wastewater sample; and (c) repeating (a)-(b) 2 or more times to monitor the presence of antibody to the antigen of SARS-CoV-2 or variant thereof within the population.

The present invention is explained in greater detail in the figures herein and the description set forth below.

### BRIEF DESCRIPTION OF THE FIGURES

FIG. 1 shows a generalized timeline of antibody waxing and waning in the human immune response.

FIG. 2 shows anti-spiked(S) and anti-nucleocapsid (N) IgG timing in wastewater collected at the UNC-Charlotte Student Union from April to December 2021.

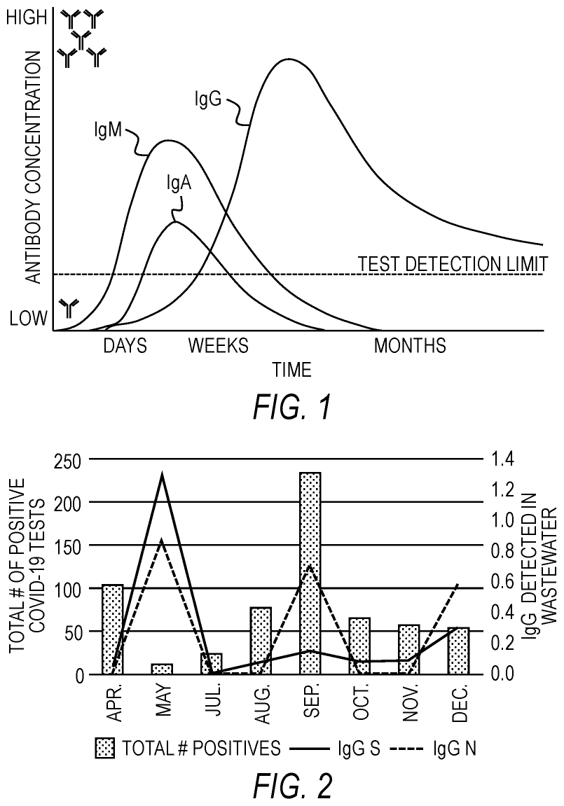

### DETAILED DESCRIPTION OF THE INVENTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety. In case of a conflict in terminology, the present specification is controlling.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR § 1.822 and established usage. See, e.g., PatentIn User Manual, 99-102 (November 1990) (U.S. Patent and Trademark Office).

Except as otherwise indicated, standard methods known to those skilled in the art may be used for cloning genes, amplifying and detecting nucleic acids, and the like. Such techniques are known to those skilled in the art. See, e.g., Green et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

### Definitions

The following terms are used in the description herein and the appended claims.

As used herein, “a,” “an,” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

As used herein, the terms “protein” and “polypeptide” are used interchangeably and encompass both peptides and proteins, unless indicated otherwise.

The term “antibody” as used herein refers to an immunoglobulin which specifically binds to and is thereby defined as complementary with a particular spatial and polar organization of another molecule. The antibody can be monoclonal, polyclonal, or a recombinant antibody. Antibodies may include a complete immunoglobulin or fragment thereof, which immunoglobulins include the various classes and isotypes, such as IgA (e.g., IgAQ1 and IgA2), IgD, IgE, IgG (e.g., IgG1, IgG2a, IgG2b, IgG3 and IgG4), IgM, IgY, etc. Fragments thereof may include Fab, Fv and F(ab′)2, Fab′, scFv, and the like. In some embodiments, the antibody is a human or animal antibody. In other embodiments, the antibody is an IgA (e.g., IgAQ1 and IgA2) or IgG (e.g., IgG1, IgG2a, IgG2b, IgG3 and IgG4).

The term “antigen” or “antigen of interest” as used herein refers to any entity that binds to an antibody. An antigen of interest is capable of inducing an immune response, in particular antibody production, in a subject upon administration of the antigen to the subject in a sufficient amount to induce the immune response. Antigens may be proteins, peptides, antibodies, small molecules, lipid, carbohydrates, nucleic acid, and allergens. An antigen may be in its pure form or in a sample in which the antigen is mixed with other components. In some embodiments, the antigen of interest is from a pathogen, e.g., a virus, bacterium or fungus. In some embodiments, an antigen of interest is from a vaccine.

With reference to antibody-antigen interactions, the term “bind” or “binding” refers to the interaction between an antibody and a cognate antigen that is typically mediated by an affinity region of the antibody. An antibody “specifically binds” a target antigen such that the antibody exhibits specific or preferential recognition for a particular target antigen compared to substantially less recognition of other molecules. In some embodiments, a specific binding interaction will discriminate between desirable and undesirable target antigens in a sample, typically more than about 10-fold to 100-fold or more (e.g., more than about 1000-fold or 10,000-fold).

The term “immune status” refers to the antibody response of a subject to a foreign substance and/or pathogen. During an active immune response, e.g., induced either by vaccination or natural infection, antigen-specific B cells are activated and clonally expand, and antibodies specific to the antigen are secreted by the B cells. The presence and/or quantity of antibodies produced is indicative of the level or degree to which the subject raises an immune response to the foreign substance and/or pathogen.

As used herein the term “pathogen” refers to an organism, including a microorganism, which causes disease in another organism (e.g., animals) by directly infecting the other organism, or by producing agents that causes disease in another organism (e.g., bacteria that produce pathogenic toxins and the like). As used herein, a pathogen may include, but is not limited to a bacterium, a parasite, a fungus, a viroid or a virus, or any combination thereof, wherein each pathogen is capable, either by itself or in concert with another pathogen, of eliciting disease in vertebrates including but not limited to mammals, and including but not limited to humans. As used herein, the term “pathogen” also encompasses microorganisms which may not ordinarily be pathogenic in a non-immunocompromised host.

Bacterial pathogens include Gram-positive bacteria and Gram-negative bacteria. A bacterial pathogen may be a species of Staphylococcus, e.g. Staphylococcus aureus, Staphylococcus epidermidis: Enterococcus spp., e.g. Enterococcus faecalis; Streptococcus pyogenes; Listeria spp.; Pseudomonas spp.; Mycobacterium spp., e.g. Mycobacterium tuberculosis: Enterobacter spp.; Campylobacter spp.; Salmonella spp, Streptococcus spp., e.g Streptococcus Group A or B. Streptococcus pneumoniae; Helicobacter spp., e.g. Helicobacter pylori; Neisseria spp., e g. Neisseria gonorrhea, Neisseria meningitidis; Borrelia burgdorferi; Shigella spp., e.g. Shigella flexneri; Escherichia coli: Haemophilus spp., e.g. Haemophilus influenzae: Chlamydia spp., e.g. Chlamydia trachomatis, Chlamydia pneumoniae, Chlamydia psittaci; Francisella nidarensis: Bacillus spp., eg. Bacillus anthracis; Clostridia spp., e.g Clostridrium botulinum; Yersinia spp., e.g. Yersinia pestis; Treponema spp.; or Burkholderia spp.; e.g. Burkholderia mallei and Burkholderia pseudomallei.

A parasitic pathogen may be a species of Trypanosoma, e.g., Trypanosoma cruzi, Trypansosoma brucei; Leishmania spp.; Giardia spp.; Trichomonas spp.; Entamoeba spp.; Naegleria spp.; Acanthamoeba spp.; Schistosoma spp.; Plasmodium spp.; Crytosporidium spp.; Isospora spp.; Balantidium spp.; Loa; Ascaris lumbricoides; Dirofilaria immitis; or Toxoplasma ssp., e.g., Toxoplasma gondii.

A fungal pathogen may be a species of the genus Candida, e.g. C. albicans; Epidermophyton spp.; Exophiala spp.; Microsporum spp.; Trichophyton spp., e.g. T. rubrum and T. interdigitale; Tinea spp.; Aspergillus spp.; Blastomyces spp.; Blastoschizomyces spp.; Coccidioides spp.; Cryptococcus spp.; Histoplasma spp.; Paracoccidiomyces spp.; Sporotrix spp.; Absidia spp.; Cladophialophora spp.; Fonsecaea spp.; Phialophora spp.; Lacazia spp.; Arthrographis spp.; Acremonium spp.; Actinomadura spp.; Apophysomyces spp.; Emmonsia spp.; Basidiobolus spp.; Beauveria spp.; Chrysosporium spp.; Conidiobolus spp.; Cunninghamella spp.; Fusarium spp.; Geotrichum spp.; Graphium spp.; Leptosphaeria spp.; Malassezia spp.; Mucor spp.; Neotestudina spp.; Nocardia spp.; Nocardiopsis spp.; Paecilomyces spp.; Phoma spp.; Piedraia spp.; Pneumocystis spp.; Pseudallescheria spp.; Pyrenochaeta spp.; Rhizomucor spp.; Rhizopus spp.; Rhodotorula spp.; Saccharomyces spp.; Scedosporium spp.; Scopulariopsis spp.; Sporobolomyces spp.; Syncephalastrum spp.; Trichoderma spp.; Trichosporon spp.; Ulocladium spp.; Ustilago spp.; Verticillium spp.; or Wangiella spp.

A viral pathogen may be a virus such as Human Immunodeficiency Virus, e.g., HIV1 or HIV2; Human T Cell Leukaemia Virus, e.g., HTLV1 or HTLV2; Ebola virus; human papilloma virus, e.g. HPV-2, HPV-5, HPV-8 HPV-16, HPV-18, HPV-31, HPV-33, HPV-52, HPV-54 or HPV-56; papovavirus; rhinovirus; poliovirus; herpesviruses, e.g., herpes simplex virus (HSV) types 1 and 2, varicella-zoster virus (VZV), cytomegalovirus (CMV), Epstein-Barr virus (EBV), human herpesvirus 6 (variants A and B), human herpesvirus 7, and Kaposi's sarcoma virus or human herpesvirus 8; adenovirus, influenza virus, e.g., influenza A or influenza B; hepatitis B and C viruses; Eastern Equine Encephalitis Virus (EEEV); West Nile virus (WNE); JC virus (JCV); BK virus (BKV); Variola virus; rotavirus; and coronaviruses such as severe acute respiratory syndrome coronavirus (SARS-CoV, e.g., SARS-CoV-1 or SARS-CoV-2) or Middle East respiratory syndrome coronavirus (MERS-CoV).

SARS-CoV-2 is a coronavirus which has four major structural proteins, specifically the spike(S), envelope (E), membrane (M) and nucleocapsid (N) proteins. The N protein holds the RNA genome, while the other three structural proteins are components of the viral envelope. The S protein is responsible for allowing the virus to attach and fuse to the membrane of a host cell. It comprises an S1 domain which mediates the attachment and an S2 domain which mediates the fusion of the viral cellular membrane with the host cell. The S1 domain comprises the receptor binding domain (RBD), the binding site to the receptor angiotensin converting enzyme 2 (ACE2) on human host cells. Therefore, the RBD is a binding site of neutralizing antibodies which block the interaction between the virus and its host cells, thus conferring immunity. By contrast to SARS-CoV-1 and SARS-CoV-2, which are associated with a high mortality and severe illness, other coronaviruses exist which are associated with a mild and passing illness, such as coronaviruses 229E, NL63, OC43 and HKU1 These coronaviruses are frequently associated with common cold, in particular among children. In embodiments, the term “SARS-CoV-2” includes variants of SARS-CoV-2.

SARS-CoV-2 may be characterized by the genome deposited on GenBank under accession no. MN908947, and variants thereof having at least 80%, 85%, 90%, 91%, 92%, 93%, 94%, 95%, 96%, 97%, 98%, 99%, 99.5%, 99.8%, 99.9% or 99.99% sequence identity over the entire genome nucleotide sequence to the nucleotide sequence deposited under accession no. MN908947. Variants include, e.g., U.K. variant B.1.17, the South African variant B.1.351, the Brazilian variant P.1 and the Mink Variant from Denmark, etc.

A “vaccine” is a preparation that is used to stimulate the body's immune response against disease. Vaccines include inactivated vaccines (e.g., against Hepatitis A, influenza, polio or rabies); live-attenuated vaccines (e.g., against measles, mumps, rubella, rotavirus, smallpox, chickenpox or yellow fever); messenger RNA vaccines (e.g., against COVID-19); subunit, recombinant, polysaccharide, or conjugate vaccines (e.g., against, Hib disease, hepatitis B, human papillomavirus, whooping cough, pneumococcal disease, meningococcal disease, or shingles); toxoid vaccines (e.g., against diphtheria or tetanus), or viral vector vaccines (e.g., against COVID-19). An antigen “of a vaccine” or “derived from a vaccine” may be the antigen of the vaccine (e.g., a subunit vaccine), a component of the vaccine (e.g., an antigen on the surface of an inactivated or live-attenuated virus), or an antigen produced by the vaccine (e.g., protein expressed from an mRNA).

A “population” refers to a group of subjects (e.g., humans or animals) living in the same place. In some embodiments, a population refers to a community of people that have a common sewershed. In some embodiments, a population may be a household, campus, or facility.

“Wastewater,” also referred to as sewage, refers to the water from household or building use (such as toilets, showers, and sinks) that can contain human fecal waste, as well as water from non-household sources (such as rain and industrial use). Wastewater from a sewershed (the community area served by a wastewater collection system) is collected as it flows into a treatment plant. In some forms, the area served by the sewershed is a single building, a single building complex, a single campus, a single city block, a single neighborhood, a single community, a single city, or a single administrative district.

“Concentrating” or “to concentrate” means that the amount or concentration of an analyte of interest in a solution is increased. In some embodiments, concentration of an analyte of interest may occur by removing solvent (e.g., water) and optionally additional analytes present in the solution, while retaining at least about 70% (e.g., about 70% to about 100%, e.g., about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range or value therein) of the analyte of interest in the solution.

As used herein, the term “ultrafiltration” or “UF” refers to any technique in which a solution or a suspension is subjected to a semi-permeable membrane that retains macromolecules while allowing solvent and small solute molecules to pass through. Ultrafiltration may be used to increase the concentration of macromolecules in a solution or suspension. In a preferred embodiment, ultrafiltration is used to increase the concentration of a protein in a solution.

### Surveillance Methods

The invention presents methods for monitoring immune status in a population by surveilling wastewater for the presence and/or quantity of antibodies to an antigen of interest, e.g., an antigen of a pathogen. In one aspect, the invention provides a method of detecting the presence of an antibody to an antigen of interest within a population by contacting a wastewater sample from a population with an antigen of interest and detecting binding of the antibody to the antigen of interest. In some embodiments, one or more wastewater samples, such as an aliquot of wastewater, is collected from one or more wastewater access points (e.g., wastewater treatment plants, influent pump stations, interceptor lines, outflow from an individual facility such as a school or dormitory). In some embodiments, one or more wastewater samples may be collected by an autosampler. In some embodiments, wastewater includes blackwater, greywater, and combinations thereof. In some embodiments, the wastewater sample has a volume of at least 10 mL (e.g, 10 mL, 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1000 mL, or more). In some embodiments, the volume of the wastewater sample is in the range of 10 mL to 200 mL, or any range or value therein. In some embodiments, samples may be stored frozen (−80° C. to −20° C.), or stored at low temperature such as about 4° C., or about 2° C. to 7° C., following collection and/or transported on ice for processing at a later time, if needed. In some embodiments, the wastewater sample is fresh. In some embodiments, the wastewater sample may be clarified by filtration and/or centrifugation, e.g., in an ultracentrifuge, to remove particular matter.

In some aspects, the wastewater sample is concentrated prior to being contacted with an antigen of interest. Concentration of the wastewater sample may be by any known means, including filtration such as ultrafiltration/diafiltration or tangential flow filtration; precipitation (e.g., ethanol, ammonium sulfate, or polyethylene glycol precipitation); dialysis; electrophoresis (e.g., SDS-PAGE); and/or chromatography such as affinity chromatography, cation exchange chromatography, anion exchange chromatography and/or hydrophobic interaction chromatography. In some embodiments, proteins, in particular antibodies, in the wastewater sample are concentrated. Chromatography techniques may exploit the chemical and physical properties of proteins to separate proteins from other analytes. These chemical and physical properties may include size, isoelectric point, charge distribution, hydrophobic sites and affinity for ligands (see, e.g., Janson, J. C. & L. Ryden (eds.). (1989) Protein Purification: Principles, High Resolution Methods and Applications. VCH Publishers, Inc., New York). The various separation modes of chromatography include, e.g., ion-exchange, chromatofocusing, gel filtration (size exclusion using, e.g., SEPHADEX™ G-75), hydrophobic interaction, reverse phase, and affinity chromatography (e.g., protein-A, protein-G, antigen-affinity or anti-IgG affinity chromatography). Ion-exchange chromatography (IEX), including anion-exchange and cation-exchange chromatography separates analytes (e.g., proteins) by differences of their net surface charges. Suitable ion-exchange resins include, e.g., S-SEPHAROSE™ and DEAE.

In an example, to concentrate the wastewater sample, in particular proteins of the wastewater sample, the wastewater sample may be exposed to an immobilized reagent that binds to the proteins. Thus, the wastewater sample may be subjected to affinity chromatography wherein an immobilized reagent that binds specifically to the proteins, such as antibodies, captures the antibodies and impurities pass through the affinity chromatography column. The proteins may be subsequently eluted from the column by changing the conditions such that the proteins no longer bind to the immobilized reagent. Protein A is commonly used as an affinity ligand which may be immobilized on various supports and allows for enrichment of samples containing proteins such as antibodies comprising an Fc region. Protein A is a 41 kD cell wall protein from Staphylococcus aureas which binds with a high affinity (about 10⁻⁸ M to human IgG) to the Fc region of antibodies (Chadha et al. (1981) Preparative Biochemistry 11 (4): 467-482; Reifsnyder et al. (1996) J. Chromatography 753:73-80). Protein A may be immobilized onto a solid phase such as glass, silica, agarose or polystyrene. The solid phase may be a purification column or a discontinuous phase of discrete particles such as a controlled pore glass column or a silicic acid column or coated with a reagent (such as glycerol) which is intended to prevent nonspecific adherence of contaminants to the solid phase (U.S. Pat. No. 6,870,034). Examples of protein A-based affinity chromatography resins include PROSEP® Ultra media, POROS™ 50A polystyrene resin and rProtein A SEPHAROSE™ Fast Flow agarose resin.

In some aspects, concentration of the wastewater sample is by ultrafiltration. Ultrafiltration is a membrane filtration and fluid exchange technique based on the size of the protein molecule of interest, e.g. antibody Membrane Diafiltration (DF) is a particular type of ultrafiltration in which a buffer is added continuously to the retentate, allowing the protein of interest to be concentrated continuously, while displacing the protein of interest from solvent system to another Ultrafiltration membranes comprise pores which are smaller than the size of the analyte (e.g., protein) to be concentrated. When passing a liquid containing the analyte through such a filter, the liquid passes through as a filtrate while the analyte cannot pass and forms a retentate. In some embodiments, the ultrafiltration membrane has a molecular weight cut-off in the range of about 10 k Da to about 100 kDa (e.g., about 10, 20, 30, 40, 50, 60, 70, 80, 90, or 100 kDa). In some embodiments, the ultrafiltration membrane has a molecular weight cut-off of about 30 kDa. In some embodiments, the retentate solution is analyzed to detect analyte present. For example, the wastewater sample may be concentrated using a commercially available protein concentration filter, for example, an AMICON® or Millipore PELLICON® ultrafiltration unit. A protease inhibitor such as PMSF may be included in any of the foregoing steps to inhibit proteolysis and antibiotics may be included to prevent the growth of contaminants.

In some embodiments, the wastewater sample is concentrated at least about 10-fold (e.g., about 10-fold to about 100-fold, e.g., about 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41- , 42-, 43-, 44-, 45-, 46-, 47-, 48-, 49-, 50-, 51-, 52-, 53-, 54-, 55-, 56-, 57-, 58-, 59-, 60-, 61-, 62- , 63-, 64-, 65-, 66-, 67-, 68-, 69-, 70-, 71-, 72-, 73-, 74-, 75-, 76-, 77-, 78-, 79-, 80-, 81-, 82-, 83- , 84-, 85-, 86-, 87-, 88-, 89-, 90-, 91-, 92-, 93-, 94-, 95-, 96-, 97-, 98-, 99-, or 100-fold, or any range or value therein) as compared to the unconcentrated wastewater sample. In some embodiments, the wastewater sample is concentrated at least about 100-fold. Concentration of the wastewater sample may be assessed by volume or amount of analyte present in the sample.

To detect or measure the presence and optionally amount of an antibody that binds to an antigen of interest, the wastewater sample (e.g., a concentrated wastewater sample) is contacted with an antigen of interest for a period of time sufficient to allow antibodies present in the wastewater sample to bind to the antigen of interest and form a complex. In some embodiments, the antibody is an IgG or IgA. In some embodiments, the antigen of interest is from a pathogen, e.g., a virus, a parasite, a fungus or a bacterium. In some embodiments, the antigen of interest is from a virus, e.g., a coronavirus. In some embodiments, the antigen of interest is an antigen of SARS-CoV-2 or variant thereof. In some embodiments, the antigen of interest is a spike protein and/or nucleocapsid protein of a virus. In some embodiments, the antigen of interest is a spike protein and/or nucleocapsid protein of SARS-CoV-2 or variant thereof. In some embodiments, the antigen of interest is from a vaccine. In some embodiments, the antigen of interest is the SARS-CoV-2 spike protein from a vaccine.

The presence and optionally amount of the antibody in the wastewater sample may be determined using an immunoassay such as an immunodiffusion assay, immunoelectrophoretic assay, light scattering immunoassay, agglutination assay, or labeled immunoassay such as a radiolabeled immunoassay, enzyme immunoassay such as colorimetric assay, chemiluminescence immunoassay or immunofluorescence technique. The person skilled in the art is familiar with these methods, which are also described in the state of the art, for example in Zane (2001) Immunology-Theoretical & Practical Concepts in Laboratory Medicine, W. B Saunders Company.

In an exemplary embodiment, the test format is an enzyme-linked immunosorbent assay (ELISA). In some embodiments, the assay is a sandwich ELISA. In an exemplary sandwich ELISA, at least one antigen of interest (e.g., an antigen of SARS-CoV-2) may be immobilized onto a selected surface exhibiting protein affinity, such as a well in a polystyrene microtiter plate Subsequently, the wastewater sample is added to the wells. After binding and/or washing to remove non-bound materials, biotinylated anti-human immunoglobulin antibodies having binding specificity for the C-region of human antibodies are introduced into the wells and allowed to bind to any human antibodies (e.g., IgA and/or IgG) bound to the target antigen of interest. Detection of the presence of the human antibodies is by reacting a streptavidin-conjugated horseradish peroxidase (or other suitable enzyme) bound to the biotin moieties with a suitable substrate such as 3,3,5,5′-tetramethylbenzidine (TMB). The amount of the reaction product from this substrate may be qualitatively or quantitatively detected by measuring its optical absorbance (for example, with TMB as the substrate at 415 nm absorbance).

As will be understood by those of skill in the art, notwithstanding individual features (e.g., the confirmatory steps described herein), in general, ELISAs have certain features in common, such as coating, incubating and binding, washing to remove non-specifically bound species, and detecting the bound immune complexes.

In coating a plate with an antigen of interest, the wells of the plate will generally be incubated with a solution of the antigen of interest, either overnight or for a specified period of hours. A coating buffer may be a sodium phosphate/bovine serum albumin (BSA) coating buffer or another suitable art-known coating buffer. The wells of the plate will then be washed to remove incompletely adsorbed material. Any remaining available surfaces of the wells may then “coated” with a nonspecific protein that is antigenically neutral with regard to the test sample. This protein may be bovine serum albumin (BSA), casein or solutions of milk powder, etc. The coating allows for blocking of non-specific adsorption sites on the immobilizing surface and thus reduces the background caused by non-specific binding of antibodies onto the surface.

In the ELISA method of the disclosure, a secondary or tertiary detection means may be used. When using a secondary or tertiary detection methods, after binding of an antigen of interest to the well, coating with a non-reactive material to reduce background (e.g., with blocking buffer such as Tris-sucrose blocking buffer or other art-recognized blocking buffer), and washing to remove unbound material, the immobilizing surface is contacted with the wastewater sample to be tested under conditions effective to allow a complex (e.g., anti-SARS-CoV-2-specific antibody/SARS-CoV-2-specific antigen) formation. Detection of this immune complex then requires a labeled (biotin) secondary binding antibody, and a third binding ligand, e.g., streptavidin-horseradish peroxidase.

The term “under conditions effective to allow immune complex formation” as used herein refers to the conditions such as, but not limited to, diluting the antigens and/or antibodies with solutions such as BSA, bovine gamma globulin (BGG), phosphate-buffered saline (PBS)/polysorbate 20, PBS with casein and polysorbate 20, or PBS/BSA buffer with polysorbate 20. Various other art-known assay diluents can be used in the methods of the invention. These added agents also tend to assist in the reduction of non-specific background.

The “suitable” conditions as used herein, means that the incubation is at a temperature or for a period of time sufficient to allow effective binding. Incubation steps are typically from about 1 to 2 or about 1 to 4 hours, at temperatures preferably on the order of 25° C. to 27° C., or may be overnight at about 4° C. Various art-known assay temperature and timing parameters can be used in the methods of the invention.

To provide a detecting means, the secondary binding antibody may have an associated detectable label. In certain embodiments, the detectable label is an enzyme that will generate color development upon incubating with an appropriate chromogenic substrate. Thus, for example, one may contact or incubate the immune complex with a urease-, glucose oxidase-, alkaline phosphatase-, hydrogen peroxidase-conjugated antibody, or other conjugated secondary binding antibody for a period of time and under conditions that favor the development of further immune complex formation (e.g., incubation for 2 hours at room temperature in a PBS-containing solution such as PBS-polysorbate 20).

It also will be understood by those of skill in the art that one or more positive and negative controls may be used in the methods of the invention. A positive control sample may be a sample that contains a predetermined amount of a human IgA and/or IgG antibody known to bind an antigen of interest (e.g., a SARS-CoV-2 antigen). Control samples may be reacted in parallel with and under the same conditions as the test samples of the assay and provide a measure of the function of the assay.

A negative control sample may be a sample known not to include an antibody that is known to bind to an antigen of interest (e.g., a SARS-CoV-2 antigen). One of skill will understand how to use positive and negative control reactions and samples in an ELISA to ascertain and validate the functionality of the solutions and/or substrates and/or protocol used in the assay. A positive control may include a known amount of a human IgA and/or IgG antibody. A negative control may be a sample that is known to not include a human IgA and/or IgG antibody. Such a negative control, when treated under the same conditions as the test sample, will demonstrate that the binding detected in a test sample arises from the test sample and is not due to contamination of assay components or other factor not associated with the test sample.

It should be appreciated that the wastewater sample (e.g., concentrated wastewater sample) may be diluted before being assayed (e.g., 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, or 32-fold, and including higher or lower fold values or any fold value in between). In one embodiment, a reference sample containing a threshold amount of reference antibody may be diluted by the same amount as the test sample being tested so that the signal obtained for the test sample can be compared directly to the signal obtained for the reference sample. In some embodiments, more than one reference sample is used.

In some aspects, the methods of the disclosure can be performed in a qualitative format, which detects the presence or absence of at least one of a human IgA and/or IgG antibody in the sample and which specifically binds an antigen of interest (e.g., a SARS-CoV-2 antigen). In some aspects, the methods of the disclosure can be performed in a quantitative format, which provides a measurement of the quantity of at least one of a human IgA and/or IgG antibody in the sample that specifically binds an antigen of interest (e.g., a SARS-CoV-2 antigen). Results may be reported either as a qualitative result (positive or negative), using specific mean channel cut-off or as semi-quantitative values by dividing the mean channel fluorescence of the positive sample by the mean channel of the negative control. This creates the potential for monitoring titers of the specific antibody. Quantitative results may also be incorporated by using known multiple positive control standards, which may form concentration curves when plotted on a graph of result versus concentration value.

In some embodiments, the presence, absence, or quantity of an antibody to an antigen of interest may be determined once, intermittently, or continuously over a period of time to monitor the presence of the antibody to the antigen of interest within the population. Accordingly, in some embodiments, the steps of contacting a wastewater sample from a population with an antigen of interest and detecting binding of the antibody to the antigen of interest may be repeated 2 or more times (e.g. 2, 3, 4, 5, 6, 7, 8, 9, 10, 11, 12, 13, 14, 15, 16 or more times). In some embodiments, the presence, absence or quantity of an antibody to an antigen of interest may be determined hourly (e.g., every hour, every two hours, every three hours, every four hours, every five hours, every six hours, every seven hours, every eight hours, every nine hours, every 10 hours, every 11 hours, every 12 hours, every 13 hours, every 14 hours, every 15 hours, every 16 hours, every 17 hours, every 18 hours, every 19 hours, every 20 hours, every 21 hours, every 22 hours every 23 hours, or every 24 hours), daily (e.g., every day, every two days, every three days, every four days, every five days, every six days, or every seven days), weekly (e.g., every week, every two weeks, every three weeks, or every four weeks), monthly (e.g., every month, every two months, every three months, every four months, every five months, every six months, every seven months, every eight months, every nine months, every 10 months, every 11 months, or every 12 months), yearly or after a specified period, e.g., after detection of a pathogen in a population or after the start of a vaccination campaign.

Wastewater surveillance approaches for viruses such as SARS-CoV-2 focus on measurements of viral abundance that reflect viral shedding by already-infected individuals. Such data is invaluable for identifying infection outbreaks and informing the needs of immediate public health responses. As antibody levels remain high weeks to months after infection in individuals, the continual detection of antibodies in combination with pathogen surveillance provides for proactive public health planning and actions. For example, antibody detection in wastewater enables agencies to address a population-wide waning of SARS-CoV-2 immunity with vaccination campaigns before infections become common. Accordingly, in an embodiment of this invention, the methods further include the step of detecting and/or quantifying the amount of the pathogen present in the wastewater sample, e.g., using known antibody-based detection and/or nucleic acid-based detection means.

In some aspects, at least one antigen is used in the methods of this invention. In other aspects, more than one antigen of interest is used, e.g., 2, 3, 4, 5, 6, or more antigens are used to detect the presence of multiple antibodies in wastewater. By way of illustration, capture of SARS-CoV-2 anti-nucleocapsid (N) and anti-spike(S) antibodies may be used to differentiate recent circulating infection from waning of vaccine-mediated immunity. This is possible since SARS-CoV-2 vaccines such as mRNA-1273 and BNT1262b elicit an immune response to the spike protein in isolation. In contrast, natural infection with SARS-CoV-2 also elicits immune responses to other components of the virus, e.g., anti-N antibodies. Therefore, the absence of anti-N paired with high anti-S levels would reveal a signature of a durable vaccine response, while the absence of both would reveal a population that is potentially at risk for future infection.

In another aspect of this invention is provided a method of determining immune status of a population to a pathogen comprising (a) contacting a wastewater sample from a population with at least one antigen from the pathogen; and (b) detecting binding of antibodies present in the wastewater sample to the at least one antigen from the pathogen, thereby determining the immune status of the population to the pathogen. The steps of contacting a wastewater sample from a population with at least one antigen from the pathogen and detecting binding may be carried out as generally described above. In one embodiment, the pathogen is a bacterium, parasite, fungus, or virus, e.g., a coronavirus such as SARS-CoV-2 or variant thereof. In some embodiments of this method, the at least one antigen is the spike protein and/or nucleocapsid protein. In other embodiments, the at least one antigen is from a vaccine. In some embodiments, the steps of the method are repeated 2 or more times to monitor the population's immune status to the pathogen over a period of time, e.g., days, weeks, months or years.

In a further aspect, this invention provides a method of monitoring the presence of an antibody to an antigen of SARS-CoV-2 or variant thereof within a population, comprising the steps of (a) concentrating a wastewater sample from a population; (b) measuring the presence and optionally amount of an antibody that binds to an antigen of SARS-CoV-2 or variant thereof in the wastewater sample; and (c) repeating (a)-(b) 2 or more times to monitor the presence of the antibody to the antigen of SARS-CoV-2 or variant thereof within the population. The steps of concentrating a wastewater sample from a population and measuring the presence and optionally amount of an antibody that binds to an antigen of SARS-CoV-2 or variant thereof may be carried out as generally described above.

In some embodiments, the concentrated wastewater sample is added to the wells of a first multi-well plate, the wells of which have been coated with a SARS-CoV-2 N protein or antigenic fragments thereof, a SARS-CoV-2 S protein or antigenic fragments thereof, or a combination of SARS-CoV-2 N protein and SARS-CoV-2 S protein either as native polypeptides and/or antigenic fragments thereof. In some embodiments, the wells of the first multi-well plate may be coated with SARS-CoV-2 N protein or antigenic fragments thereof, and a fragment of the SARS-CoV-2 S protein comprising the Receptor Binding Domain (RBD) thereof. Further provided is a second multi-well plate wherein the wells are coated with serum albumin. In some embodiments, the SARS protein coated and albumin-coated wells are of the same multi-well plate. In some embodiments, the concentrated wastewater sample is diluted and added to at least two wells of a first multi-well plate, one well of which has been coated with the SARS-CoV-2 N protein or antigenic fragments thereof, and the other well of which has been coated with a fragment of the SARS-CoV-2 S protein comprising the RBD thereof.

In some embodiments, the SARS-CoV-2 antigen is the spike protein of the virus. The full-length spike protein has the amino acid sequence of SEQ ID NO:1 (Accession No: QHD43416). A fragment of the SARS-CoV-2 S protein encompassing the RBD includes, but not is not limited to the amino acids R319-F541 (SEQ ID NO:2) or antigenic fragments thereof that have affinity with, and are bound by, an anti-SARS-CoV-2-specific antibody. The methods of the disclosure may further be adapted to use the spike protein region 2 (amino acids M697-P1213 (SEQ ID NO: 3)), or antigenic fragments thereof. Determination of antigenic fragments that can be useful in the methods of the disclosure can be obtained and confirmed to bind anti-SARS-CoV-2 antibodies by methods well-known to those of skill in the art.

In some embodiments, the SARS-CoV-2 antigen is the nucleocapsid protein, or antigenic fragments thereof, of SARS-CoV-2. The full-length nucleocapsid protein has the amino acid sequence of SEQ ID NO:4 (Accession No: QHD43423).

In some embodiments, the disclosure encompasses a method of monitoring the presence of antibodies to an antigen of SARS-CoV-2 or variant thereof within a population, the method comprising: (a) obtaining a wastewater sample from a population; (b) removing particulate matter from the wastewater sample by centrifugation; (c) concentrating the wastewater sample from a population by ultrafiltration, wherein the wastewater sample comprises anti-SARS-CoV-2 antibodies; (d) adding the concentrated wastewater sample to at least one well of a multi-well plate, wherein each well of the multi-well plate is coated with SARS-CoV-2 spike protein and/or SARS-CoV-2 nucleocapsid protein; (e) incubating the concentrated wastewater sample and SARS-CoV-2 spike protein and/or SARS-CoV-2 nucleocapsid protein under conditions effective to allow immune complex formation; (f) washing unbound material from the well; (g) adding a biotinylated anti-human IgA antibody or a biotinylated anti-human IgG antibody to the well having the immune complex; (h) incubating the well for a period effective to allow the biotinylated anti-human IgA antibody or biotinylated anti-human IgG antibody to bind to the immune complex; (i) washing the well to remove unbound biotinylated anti-human IgA antibody or biotinylated anti-human IgG antibody; (j) adding a horseradish peroxidase (HRP)-streptavidin conjugate and an HRP substrate to the well from step (i), thereby generating a light detectable product; (k) measuring the light absorbance of the product from step (j) for the well; (l) determining the presence and optionally amount of anti-SARS-CoV-2 spike IgA and/or IgG and/or anti-SARS-CoV-2 nucleocapsid IgA and/or IgG in the wastewater sample from the measured absorbance of the well having the bound immune complex (e.g., minus the measured absorbance of a control well or using a standard curve); (m) repeating steps (a)-(l) 2 or more times; and (n) comparing the presence and optionally quantity of anti-SARS-CoV-2 spike IgA and/or IgG and/or anti-SARS-CoV-2 nucleocapsid IgA and/or IgG in the wastewater sample over time thereby monitoring the presence of antibodies to SARS-CoV-2 spike and/or nucleocapsid antigens within the population.

In some embodiments, the method may further comprise repeating the steps (d)-(l) for each of the SARS-CoV-2 spike protein and/or SARS-CoV-2 nucleocapsid protein. In some embodiments, the method may further comprise repeating the steps (g)-(l) for each of the biotinylated anti-human IgA antibody and the biotinylated anti-human IgG antibody, or the antigen-binding fragments thereof. In some embodiments, the method may further comprise determining the relative levels of one or both of human IgA and IgG bound to the SARS-CoV-2 proteins or fragments thereof. In some embodiments, the HRP substrate may be 3,3,5,5′-tetramethylbenzidine. In some embodiments, the method may be a high-throughput assay. In some embodiments, the bound SARS-CoV-2 spike protein may comprise amino acid residues Arg319 to Phe541 (SEQ ID NO: 2), which encompass the receptor-binding domain (RBD). In some embodiments, the wastewater sample is monitored for the presence or absence of one or more variants of SARS-CoV-2, e.g., delta and/or omicron variants, using antigens specific to the one or more variants.

Wastewater samples may aggregate antibodies from a few to hundreds or thousands of individuals and may be used to provide a direct interpretation of antibody levels detected and immune status of a population. COVID-19 wastewater surveillance projects that submit to the Centers for Disease Control National Wastewater Surveillance System gather site metadata describing water flow rates and sewershed populations contributing to the collection point signal. Where that information is available, ELISA optical density or titer measurements may be extrapolated to a bulk copies per person measurement, in a way that is analogous to the handling of viral copy number data (Soller et al. (2022) J. Water Health 20:1197-1211). Because the methods of the invention are able to detect antibodies in frozen archived wastewater samples, the SARS-CoV-2 pandemic may be retrospectively analyzed and modeled to assess the relationship of antibody responses to traditional serology, case trends, and other parameters. Leveraging existing samples collected throughout the COVID-19 pandemic, historic longitudinal studies may be conducted that simultaneously quantify concentrations of antibodies and integrate that information with viral load and case data for the corresponding sewershed population.

Accordingly, in some embodiments, the methods of the present invention may further include the step of detecting and/or quantifying the amount of pathogen (e.g., bacterium, parasite, fungus, or virus such as SARS-CoV-2 or variant thereof) present in the wastewater sample. Bacterial, parasitic and fungal pathogens may be isolated from wastewater by centrifugation and/or filtration. Viral pathogens may be concentrated from wastewater with polyethylene glycol (PEG), flocculation and filtration using positively charged membranes, monolithic adsorption, and/or glass wool filtration. The presence and/or quantity of a pathogen may be determined by detecting one or more proteins and/or nucleic acids of the pathogen. In some embodiments, a protein present on the surface of a pathogen is detected. In some embodiments, a protein of a pathogen is detected using an antibody that specifically binds the protein of the pathogen. In some embodiments, one or more proteins of the pathogen are detected using an immunoassay as described herein.

For nucleic acid detection, the nucleic acids may be extracted using reagents such as TRIZOL® (guanidinium thiocyanate with phenol/chloroform extraction) and commercial kits such as QIAAMP® Viral RNA Mini Kit or POWER VIRAL® Environmental RNA/DNA Isolation Kit (both from Qiagen). Any suitable assay for detecting and quantifying nucleic acids of a pathogens in wastewater may be used including, but not limited to sequencing and/or amplification techniques based on PCR, e.g., classical PCR, real time quantitative PCR (qPCR), and reverse transcriptase quantitative PCR (RT-qPCR), integrating cell culturing of the virus inoculum from the environment followed by PCR (ICC PCR), concentrating virus particles in an environmental sample via immunomagnetic separation prior to PCR (IMS PCR), or digital PCR. In some embodiments, real time qPCR combined with reverse transcription (qRT-PCR) is used. In the case of SARS-CoV-2, real time PCR assays generally target the N and E genes on the genomic RNA, though other regions of the genome were also targeted in some cases such as ORF1a and ORF1b (RdRp gene) and the S gene. In some aspects, the CDC 2019-Novel Coronavirus (2019-nCoV) Real-Time RT-PCR Diagnostic Panel (Catalog #2019-nCoVEUA-01) and associated protocol is used. Exemplary primers and probes used in the detection of SARS-CoV-2 virus are listed in Table 1.

TABLE 1
SARS-CoV-2 primers

| | | | SEQ ID |
|---|---|---|---|
| Gene | Oligonucleotide | Sequence | NO: |
| ORFlab | Forward Primer | CTAGGACCTCTTTCTGCTCA | 5 |
| | Reverse Primer | ACACTCTCCTAGCACCATCA | 6 |
| S | Forward Primer | CCCTGTTGCTATTCATGCAG | 7 |
| | Reverse Primer | CCCTATTAAACAGCCTGCAC | 8 |
| S | Forward Primer | CCTACTAAATTAAATGATCTCTGCTTTACT | 9 |
| | Reverse Primer | CAAGCTATAACGCAGCCTGTA | 10 |
| E | Forward Primer | GGAAGAGACAGGTACGTTAA | 11 |
| | Reverse Primer | AAGGTTTTACAAGACTCACG | 12 |
| E | Forward Primer | ACAGGTACGTTAATAGTTAATAGCGT | 13 |
| | Reverse Primer | ATATTGCAGCAGTACGCACACA | 14 |
| N | Forward Primer | CCTCTTCTCGTTCCTCATCA | 15 |
| | Reverse Primer | CCTGGTCCCCAAAATTTCCT | 16 |
| N1 | Forward Primer | GACCCCAAAATCAGCGAAAT | 17 |
| | Reverse Primer | TCTGGTTACTGCCAGTTGAATCTG | 18 |
| | Probe | /56-FAM/ACCCCGCATTAC | 19 |
| | | GTTTGGTGGACC/36-TAMSp/ | |
| N2 | Forward Primer | TTACAAACATTGGCCGCAAA | 20 |
| | Reverse Primer | GCGCGACATTCCGAAGAA | 21 |
| | Probe | /56-FAM/ACAATTTGCCC | 22 |
| | | CCAGCGCTTCAG/36-TAMSp/ | |
| N3 | Forward Primer | GGGAGCCTTGAATACACCAAAA | 23 |
| | Reverse Primer | TGTAGCACGATTGCAGCATTG | 24 |
| | Probe | /56-FAM/AYCACATTGGCA | 25 |
| | | CCCGCAATCCTG/36-TAMSp/ | |
| RdRp | Forward Primer | CATCTCACTTGCTGGTTCCT | 26 |
| | Reverse Primer | CCTTAATAGTCCTCACTTCTCTC | 27 |

56-FAM, 5′ 6-FAM (Fluorescein); 36-TAMSp, 3′ TAMRA™.

One of skill in the art will further appreciate that any or all steps in the methods of the invention could be implemented by personnel or, alternatively, performed in an automated fashion. That is, the methods can be performed in an automated, semi-automated, or manual fashion. Furthermore, the methods disclosed herein can also be combined with other methods known or later developed to permit a more accurate determination of the immune status of a population to an antigen of interest.

To facilitate the detection and or quantification of an antibody to an antigen of interest (e.g., SARS-CoV-2 spike and/or nucleocapsid protein) and/or pathogen (e.g., SARS-CoV-2 or variant thereof), another aspect of the invention encompasses a kit for monitoring the presence of an antibody to an antigen of interest (e.g., an antigen of SARS-CoV-2 or a variant thereof) within a population, wherein the kit comprises a substrate coated with the antigen of interest, or an antibody-binding fragment thereof. In some aspects, the antigen of interest is an antigen from a pathogen such as a virus, fungus, bacterium or parasite. In some embodiments, the antigen of interest is from a virus. In some embodiments, the antigen of interest is from a coronavirus. In some embodiments, the antigen of interest is a SARS-CoV-2 spike protein or fragment thereof and/or a SARS-CoV-2 nucleocapsid protein or fragment thereof. By “kit” is intended any manufacture (e.g., a package or a container) comprising at least one reagent for specifically detecting the presence of a antibody in a sample, e.g., a SARS-CoV-2-specific antibody. The kit may be promoted, distributed, or sold as a unit for performing the methods of the present invention. As used herein, the term “substrate” is any surface that supports an immunoassay. The substrate of the invention may be a solid substrate or a porous substrate, for example. In some embodiments, the substrate is a multi-well plate. In some embodiments, the substrate is a test strip of an immunodiffusion assay.

In some embodiments, the kit further includes a means for detecting the presence of a pathogen, e.g., SARS-CoV-2 or a variant thereof. In accordance with some embodiments, the kit further includes a vessel or vessels containing oligonucleotides for detecting one or more nucleic acids of the pathogen; and/or a vessel or vessels containing at least one antibody for detecting a protein of the pathogen. In some embodiments, the kit further includes a vessel or vessels containing oligonucleotides for detecting one or more nucleic acids of SARS-CoV-2 or a variant thereof; and/or a vessel or vessels containing at least one antibody for detecting a protein of SARS-CoV-2 or a variant thereof. In some embodiments, the kit further includes one or more of: (i) a vessel or vessels for collecting a wastewater sample from a sewershed; (ii) an ultrafiltration membrane (e.g., with a molecular weight cut-off of 30 kDa); (iii) a vessel or vessels containing at least one of a biotinylated anti-human IgA antibody, a biotinylated anti-human IgG antibody, or antigen-binding fragments thereof; (iv) a vessel containing a horseradish peroxidase-streptavidin conjugate; and/or (v) instructions for using reagents of the kit for monitoring disease and immune status to a pathogen such as SARS-CoV-2 or a variant thereof, within the population. In some embodiments, the protein of SARS-CoV-2 is a SARS-CoV-2 spike protein. In some embodiments, the protein of SARS-CoV-2 comprises the SARS-CoV-2 spike protein from amino acid positions Arg319 to Phe541 (SEQ ID NO: 2) of the receptor-binding domain (RBD).

The present invention is further detailed in the following examples, which are offered by way of illustration and is not intended to limit the invention in any manner. Standard techniques well known in the art or the techniques specifically described below are utilized.

### EXAMPLES

### Example 1. Material and Methods

Total IgG and IgA Detection. Twenty-four hour influent samples were collected from a South Burlington, Vermont wastewater treatment plant. IL wastewater samples were homogenized and incubated with shaking at 4° C. for one hour. The homogenate was clarified via centrifugation, 3500 rpm at 4° C. for 15 minutes, followed by 13000 rpm at 4° C. for an additional 15 minutes. The clarified sample was then filtered through a 0.2 μm filter and run on a protein-G agarose column. Fractions were collected and subsequently, a Nanodrop spectrophotometer detected a peak indicating the presence of immunoglobulins, specifically IgG. This assay was not suitable for use on a large scale due to the volume of input material required and the high cost and limited availability of protein-G agarose column material to process IL volumes. Building upon this observation, we developed a scalable protocol for quantification of IgG and IgA from wastewater using commercially available reagents.

Sample collection and processing. Samples were collected from the study areas described by Gibas et al. ((2021) Sci. Total Environ. 782:146749) and Barua et al. ((2021) Sci. Total Environ. 814:152503) following the study protocols described therein. Briefly, 24-hour composite samples were collected using ISCO portable autosamplers. For this study, three sites representative of typical wastewater surveillance sampling areas were selected. One of these is a high-traffic single building, the UNC Charlotte Student Union. The second site (Greenway) is a trunkline manhole with a sub-sewershed collection area that encompasses the entire UNC Charlotte campus. The third site is the Mallard Creek (MLC) wastewater treatment facility, which is the Charlotte Water treatment plant serving a larger area that includes the university. Samples were either processed fresh or archived following collection in several 50 mL conical tubes and stored at −80° C.

Eighty mL of wastewater was split into two 50 ml conical tubes and centrifuged for 10 minutes at 10,000×g at 20° C. Remaining supernatant liquid from centrifuged 50 mL conical tubes was poured carefully into two new 50 mL conical tubes, making sure not to pour solid pellet/particles along with supernatant. Sample supernatants can be stored at −80° C. until ready for filtration. Fifteen mL of liquid sample supernatant was poured into each 30 kD filtration tube (Millipore) and centrifuged for 60 minutes at 4000×g at 4° C. This is repeated for the entire sample to ensure sufficient concentration. Concentrated protein supernatant was removed from filtration tubes and stored at −80° C.

ELISA detection of specific SARS-Cov-2 IgG and IgA. For ELISA detection of total human IgG and total human IgA, plates were pre-coated with specific antigen to human IgG by the manufacturer (Abcam), and the developer specifications were followed without modification. Briefly, a sandwich ELISA was performed using an antibody cocktail containing both capture and detector antibodies after addition of either neat or concentrated wastewater to the plate. The detector antibody is conjugated to horseradish peroxidase (HRP). After a 60 minute incubation with shaking at room temperature, the plates were washed thrice with proprietary wash buffer followed by the addition of the substrate tetramethylbenzidine (TMB) for 10 minutes. TMB detected HRP activity on bound antibodies. After 10 minutes, proprietary stop solution was added to stop the activity of TMB and change the pigment from blue (620 nm) to yellow (450 nm). After the addition of the stop solution, the plates were immediately read on a fluorescence plate reader (Biotek Synergy LX).

For ELISA detection of SARS-CoV-2 Spike(S) protein-specific and nucleocapsid (N) protein-specific and IgG, plates were pre-treated with SARS-CoV-2 N protein or S receptor binding domain (RBD) protein by the manufacturer (Abcam), and the developer specifications were followed without modification. Briefly, concentrated wastewater was added to the pre-coated plates, and washed four times with proprietary wash buffer after 60 minutes of incubation with shaking. Biotinylated anti-human IgG was added post wash for 30 minutes with shaking and washed four times after incubation with wash buffer. HRP-conjugated streptavidin was added for 30 minutes and washed as above. Streptavidin has a high binding affinity to biotin, which was coupled to the anti-human IgG. After washing away excess HRP-streptavidin, TMB was added to the plate for 15 minutes to activate the HRP. After 15 minutes, proprietary stop solution was added to terminate the HRP/TMB reaction and the plate was read at 450 nm on a fluorescence plate reader (Biotek Synergy LX). A second set of assays was run using Abcam and Raybiotech ELISA products on the same set of input samples to ensure that the results were not platform dependent (Table 2). The Raybiotech assay was followed to the developer specifications without modification.

TABLE 2
Average triplicate OD450 readings for COVID-19 S-Protein
(S1RBD) Human IgG from Union wastewater collection site:

| Dilution | Abcam | Raybiotech |
|---|---|---|
| Neat | 0.598 | 0.658 |
| 1:2 | 0.554 | 0.638 |
| 1:4 | 0.363 | 0.33 |
| Negative Control | 0.050 | 0.047 |

*NB - per manufacturer's specifications, any value greater than twice the negative control is deemed positive.

### Example 2. Detection of Bulk Human IgG and IgA in Fresh Wastewater Samples

We initially assayed whether any antibodies were detectable in fresh wastewater by measuring the abundance of total human IgG and IgA. After concentration, bulk human IgG (Table 3) and IgA (Table 4) were both detected at concentrations that exceeded the upper detection limit of the ELISA assay at all dilutions tested.

TABLE 3
OD450 readings of total human IgG on wastewater.*

| | | Concentrated | |
|---|---|---|---|
| Standard Curve | Negative Control | Wastewater | Average Triplicate |
| (OD450) | (OD450) | (Dilution) | OD450 Readings |
| 0.928 | 0.055 | Neat | >4.000 |
| 0.503 | 0.053 | 1:2 | >4.000 |
| 0.307 | 0.059 | 1:4 | >4.000 |
| 0.166 | 0.057 | 1:8 | >4.000 |
| 0.120 | 0.055 | 1:16 | >4.000 |

*NB - per manufacturer's specifications, any reading greater than twice the negative control is deemed positive.

TABLE 4
OD450 readings of total human IgA on wastewater.*

| | | Concentrated | |
|---|---|---|---|
| Standard Curve | Negative Control | Wastewater | Average Triplicate |
| (OD450) | (OD450) | (Dilution) | OD450 Readings |
| 2.611 | 0.124 | Neat | >4.000 |
| 1.577 | 0.227 | 1:2 | >4.000 |
| 0.873 | 0.108 | 1:4 | >4.000 |
| 0.485 | 0.099 | 1:8 | >4.000 |
| 0.297 | 0.100 | 1:16 | >4.000 |
| 0.197 | 0.103 | 1:32 | >4.000 |

*NB - per manufacturer's specifications, any reading greater than twice the negative control is deemed positive.

Consistent with classic serology, our approach yields ELISA assays displaying a color change in the positive sample that is visible to the eye, and can also be quantified as the optical density at 450 nm (OD450) using a plate reader (Tables 3 and 4). In the case of this example, the lower detection limit used in this experiment was 2× the negative control signal, as recommended by the manufacturer. For IgG, all dilutions of the neat sample by less than 1:16 gave a signal above background (Table 3) and for IgA, all dilutions by less than 1:32 gave a signal above background by the manufacturer's definition. These OD450 readings correspond to 5.08 ng/ml of IgG and 1.52 ng/ml of IgA in concentrated wastewater. For comparison, serum levels of total human IgG and IgA are known to be 8-18 mg/ml and 0.4 to 2.2 mg/ml respectively, or approximately 1000-fold greater (Rose (1997) Manual of Clinical Laboratory Immunology. (ASM Press).

### Example 3. Measurement of Anti-SARS-CoV-2 Spike Protein Antibodies

Given the ability to detect the presence of bulk human IgG and IgA, we next assayed specific human anti-SARS-CoV-2 spike(S) protein IgG and IgA in concentrated samples. We used both fresh samples, and frozen samples from two timepoints in an archived longitudinal series collected during the SARS-CoV-2 pandemic. We found that both anti-S IgG (Table 5) and anti-S IgA (Table 6) antibodies were detectable in quantities within or over the range of detection of the ELISA assay. This was true for fresh samples and for frozen samples.

TABLE 5
Average triplicate OD450 readings for COVID-19 S-Protein (S1RBD) Human IgG.*

| | Union Fresh | Union Frozen | Greenway Fresh | Greenway Frozen | MLC Fresh | MLC Frozen |
|---|---|---|---|---|---|---|
| Dilution | (October 2022) | (February 2022) | (October 2022) | (February 2022) | (October 2022) | (June 2020) |
| Neat | 2.58 | 2.36 | 0.971 | 0.519 | 0.599 | 0.088 |
| 1:2 | 1.42 | 2.04 | 1.09 | 0.223 | 0.291 | 0.060 |
| 1:4 | 0.705 | 1.21 | 0.972 | 0.135 | 0.189 | 0.061 |
| 1:8 | 0.391 | 0.722 | 0.695 | 0.098 | 0.163 | 0.059 |

*NB - per manufacturer's specifications, any value greater than twice the negative control is deemed positive. Average Negative Control OD450: 0.053.

TABLE 6
Average triplicate OD450 readings for COVID-19 S-Protein (S1RBD) Human IgA.*

| | Union Fresh | Union Frozen | Greenway Fresh | Greenway Frozen | MLC Fresh | MLC Frozen |
|---|---|---|---|---|---|---|
| Dilution | (October 2022) | (February 2022) | (October 2022) | (February 2022) | (October 2022) | (June 2020) |
| Neat | 2.236 | >4.000 | 0.952 | 0.865 | 0.475 | 0.107 |
| 1:2 | 1.632 | 3.772 | 0.751 | 0.704 | 0.401 | 0.103 |
| 1:4 | 0.973 | 2.889 | 0.601 | 0.516 | 0.268 | 0.100 |

*NB - per manufacturer's specifications, any value greater than twice the negative control is deemed positive. Average Negative Control OD450: 0.091.

In general, anti-S IgA was found to be present in higher abundance than anti-S IgG in recent fresh wastewater samples (Table 6). However, this was not always the case. To assess whether anti-S antibodies were detectable during the onset of the pandemic in North Carolina, we additionally assayed samples collected in June of 2020. During this time, Mecklenburg County NC had only experienced a cumulative 9294 known COVID-19 cases in a population of 1.12 million. In this case, we do not have a sufficient signal of anti-S IgG or anti-S IgA to be classified as positive. Archival samples from the June 2020 time period do, however, still produce a very high generic human IgG signal (Table 7), demonstrating that the lack of anti-S signal in June 2020 is due to its absence and not due to degradation of protein in the sample.

TABLE 7
Average triplicate readings for non-specific human
IgG and IgA in campus wastewater in June 2020.

| | |
|---|---|
| IgG | IgA |

| Dilution | | Dilution | |
|---|---|---|---|
| Factor | Average OD450 | Factor | Average OD450 |
| Neat | 3.832 | Neat | >4.000 |
| 1:2 | >4.000 | 1:2 | >4.000 |
| 1:4 | 3.392 | 1:4 | >4.000 |
| 1:8 | 2.157 | 1:8 | >4.000 |

*NB - per manufacturer's specifications, any value greater than twice the negative control is deemed positive.
IgG Average Negative Control OD450: 0.051.
IgA Average Negative Control OD450: 0.119

### Example 4. Assessing Longitudinal Trends of Antibody Concentration in Wastewater

To assess longitudinal trends in antibody concentration, we applied our approach to wastewater collected at the UNC Charlotte Student Union at monthly intervals between April and December 2021. By April 2021, vaccination campaigns had begun and traffic on campus was increasing, and by August of 2021, the on-campus resident population had been returned to normal density. We measured anti-S IgG for each sample and also extended our approach to capture anti-SARS-CoV-2 Nucleocapsid (N) IgG. Comparing the abundance of anti-S to anti-N IgG reveals parallel temporal peaks of both antibodies in May of 2021 (FIG. 2). However, there is a mismatch between the abundances of anti-S IgG and anti-N IgG in September. When contrasted to the number of known on-campus positive COVID-19 tests for each month, anti-N IgG mirrors patterns of incidence while anti-S IgG does not (FIG. 2).

The foregoing is illustrative of the present invention and is not to be construed as limiting thereof. The invention is defined by the following claims, with equivalents of the claims to be included therein. All publications, patent applications, patents, patent publications, and any other references cited herein are incorporated by reference in their entireties for the teachings relevant to the sentence and/or paragraph in which the reference is presented.

1. A method of detecting the presence of an antibody to an antigen of interest within a population comprising
(a) contacting a wastewater sample from a population with an antigen of interest, and
(b) detecting binding of the antibody to the antigen of interest, thereby detecting the antibody to the antigen of interest within the population.

2. The method of claim 1, wherein the antigen of interest is from a pathogen, optionally a bacterium, parasite, fungus, or virus.

3. (canceled)

4. The method of claim 3, wherein the virus is a coronavirus, optionally SARS-CoV-2 or variant thereof, optionally wherein the antigen of interest is the spike protein and/or nucleocapsid protein.

5-6. (canceled)

7. The method of claim 1, wherein the antigen of interest is from a vaccine.

8. The method of claim 1, wherein the step of detecting binding of the antibody to the antigen of interest comprises using an immunodiffusion assay, immunoelectrophoretic assay, light scattering immunoassay, agglutination assay, or labeled immunoassay, optionally wherein the labeled immunoassay is an enzyme-linked immunosorbent assay.

9. (canceled)

10. The method of claim 1, further comprising a prestep of concentrating the wastewater sample from the population, optionally wherein the step of concentrating the wastewater sample comprises ultrafiltration of the wastewater sample.

11. (canceled)

12. The method of claim 1, wherein the wastewater sample is fresh or frozen and/or wherein the antibody is an IgA and/or IgG antibody.

13. (canceled)

14. The method of claim 1, further comprising
(c) repeating (a)-(b) 2 or more times to monitor the presence of the antibody to the antigen of interest within the population.

15. The method of claim 1, further comprising quantifying the amount of antibody in the wastewater sample.

16. The method of claim 2, further comprising detecting and/or quantifying the amount of the pathogen present in the wastewater sample.

17. A method of determining immune status of a population to a pathogen comprising
(a) contacting a wastewater sample from a population with at least one antigen from the pathogen; and
(b) detecting binding of antibodies present in the wastewater sample to the at least one antigen from the pathogen, thereby determining the immune status of the population to the pathogen.

18. The method of claim 17, wherein the pathogen is a bacterium, parasite, fungus, or virus.

19. The method of claim 18, wherein the virus is a coronavirus, optionally wherein the coronavirus is SARS-CoV-2 or variant thereof, optionally wherein the at least one antigen is the spike protein and/or nucleocapsid protein.

20-21. (canceled)

22. The method of claim 17, wherein the at least one antigen is from a vaccine.

23. The method of claim 17, wherein the step of detecting binding of the antibody to the at least one antigen comprises using an immunodiffusion assay, immunoelectrophoretic assay, light scattering immunoassay, agglutination assay, or labeled immunoassay, optionally wherein the labeled immunoassay is an enzyme-linked immunosorbent assay.

24. (canceled)

25. The method of claim 17, further comprising a prestep of concentrating the wastewater sample, optionally wherein the step of concentrating the wastewater sample comprises ultrafiltration of the wastewater sample.

26. (canceled)

27. The method of claim 17, wherein the wastewater sample is fresh or frozen and/or wherein the antibody is an IgA and/or IgG antibody.

28. (canceled)

29. The method of claim 17, further comprising
(c) repeating (a)-(b) 2 or more times to monitor the population's immune status to the pathogen.

30. The method of claim 17, further comprising quantifying the amount of antibody in the wastewater sample and/or detecting and/or quantifying the amount of the pathogen present in the wastewater sample.

31-39. (canceled)

40. A kit for monitoring the presence of an antibody to SARS-CoV-2 or a variant thereof within a population comprising a substrate coated with a SARS-CoV-2 spike protein or fragment thereof and/or a SARS-CoV-2 nucleocapsid protein or fragment thereof.

41-43. (canceled)


SEQUENCE LISTING (27 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 214 aa, Severe acute respiratory syndrome coronavirus 2
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 3 - protein, 517 aa, Severe acute respiratory syndrome coronavirus 2
MSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 4 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - DNA, 20 bp, synthetic construct
  source 1..20: mol_type = other DNA
ctaggacctctttctgctca

SEQ ID NO: 6 - DNA, 20 bp, synthetic construct
  source 1..20: mol_type = other DNA
acactctcctagcaccatca

SEQ ID NO: 7 - DNA, 20 bp, synthetic construct
  source 1..20: mol_type = other DNA
ccctgttgctattcatgcag

SEQ ID NO: 8 - DNA, 20 bp, synthetic construct
  source 1..20: mol_type = other DNA
ccctattaaacagcctgcac

SEQ ID NO: 9 - DNA, 30 bp, synthetic construct
  source 1..30: mol_type = other DNA
cctactaaattaaatgatctctgctttact

SEQ ID NO: 10 - DNA, 21 bp, synthetic construct
  source 1..21: mol_type = other DNA
caagctataacgcagcctgta

SEQ ID NO: 11 - DNA, 20 bp, synthetic construct
  source 1..20: mol_type = other DNA
ggaagagacaggtacgttaa

SEQ ID NO: 12 - DNA, 20 bp, synthetic construct
  source 1..20: mol_type = other DNA
aaggttttacaagactcacg

SEQ ID NO: 13 - DNA, 26 bp, synthetic construct
  source 1..26: mol_type = other DNA
acaggtacgttaatagttaatagcgt

SEQ ID NO: 14 - DNA, 22 bp, synthetic construct
  source 1..22: mol_type = other DNA
atattgcagcagtacgcacaca

SEQ ID NO: 15 - DNA, 20 bp, synthetic construct
  source 1..20: mol_type = other DNA
cctcttctcgttcctcatca

SEQ ID NO: 16 - DNA, 20 bp, synthetic construct
  source 1..20: mol_type = other DNA
cctggtccccaaaatttcct

SEQ ID NO: 17 - DNA, 20 bp, synthetic construct
  source 1..20: mol_type = other DNA
gaccccaaaatcagcgaaat

SEQ ID NO: 18 - DNA, 24 bp, synthetic construct
  source 1..24: mol_type = other DNA
tctggttactgccagttgaatctg

SEQ ID NO: 19 - DNA, 24 bp, synthetic construct
  source 1..24: mol_type = other DNA
  misc_feature 1: note = 6-FAM (Fluorescein) labeled
  misc_feature 24: note = 6-TAMSp labeled
accccgcattacgtttggtggacc

SEQ ID NO: 20 - DNA, 20 bp, synthetic construct
  source 1..20: mol_type = other DNA
ttacaaacattggccgcaaa

SEQ ID NO: 21 - DNA, 18 bp, synthetic construct
  source 1..18: mol_type = other DNA
gcgcgacattccgaagaa

SEQ ID NO: 22 - DNA, 23 bp, synthetic construct
  source 1..23: mol_type = other DNA
  misc_feature 1: note = 6-FAM (Fluorescein) labeled
  misc_feature 23: note = 6-TAMSp labeled
acaatttgcccccagcgcttcag

SEQ ID NO: 23 - DNA, 22 bp, synthetic construct
  source 1..22: mol_type = other DNA
gggagccttgaatacaccaaaa

SEQ ID NO: 24 - DNA, 21 bp, synthetic construct
  source 1..21: mol_type = other DNA
tgtagcacgattgcagcattg

SEQ ID NO: 25 - DNA, 24 bp, synthetic construct
  source 1..24: mol_type = other DNA
  misc_feature 1: note = 6-FAM (Fluorescein) labeled
  misc_feature 24: note = 6-TAMSp labeled
aycacattggcacccgcaatcctg

SEQ ID NO: 26 - DNA, 20 bp, synthetic construct
  source 1..20: mol_type = other DNA
catctcacttgctggttcct

SEQ ID NO: 27 - DNA, 23 bp, synthetic construct
  source 1..23: mol_type = other DNA
ccttaatagtcctcacttctctc